(19) United States
(12) Patent Application Publication
(10) Pub. No.: US 2009/0187159 A1
(43) Pub. Date: Jul. 23, 2009

(54) CHRONICALLY IMPLANTABLE HYBRID CANNULA-MICROELECTRODE SYSTEM FOR CONTINUOUS MONITORING ELECTROPHYSIOLOGICAL SIGNALS DURING INFUSION OF A CHEMICAL OR PHARMACEUTICAL AGENT

(75) Inventors: Bradley Greger, Van Nuys, CA (US); Babak Kateb, Los Angeles, CA (US)

Correspondence Address: Law Offices of Daniel L. Dawes, 5200 Warner Blvd, Ste. 106, Huntington Beach, CA, 92649, US

(73) Assignee: California Institute of Technology, Pasadena, CA (US)

(21) Appl. No.: 12/016,181

(22) Filed: Jan. 17, 2008

(51) Int. Cl. A61M 37/00

(52) U.S. Cl. 604/503; 604/66

(57) ABSTRACT

A device for assessing the effects of diffusible molecules on electrophysiological recordings from multiple neurons allows for the infusion of reagents through a cannula located among an array of microelectrodes. The device can easily be customized to target specific neural structures. It is designed to be chronically implanted so that isolated neural units and local field potentials are recorded over the course of several weeks or months. Multivariate statistical and spectral analysis of electrophysiological signals acquired using this system could quantitatively identify electrical “signatures” of therapeutically useful drugs.

### BACKGROUND OF THE INVENTION

1. Field of the Invention

The invention relates to the field of electrophysiological implants and in particular to a chronically implantable, hybrid cannula-microelectrode device for assessing the effects of molecules on electrophysiological signals in freely behaving animals.

2. Description of the Prior Art

A variety of approaches are required to assess the neuronal mechanisms underlying behavior. Some approaches, such as localized lesions and electrical stimulation, have been used for decades to yield general information about the functions of specific brain structures or pathways. For many years, however, techniques capable of providing information about specific populations of neurons were difficult to apply to behaving animals for most investigations of the mammalian central nervous system. In fact, most recordings of electrophysiological activity in the rat brain, for example, typically are carried out while the animal is anesthetized and secured in a stereotaxic device. Although the use of these techniques with the stereotaxic preparation continues to provide new insights into brain function, the need to relate electrophysiological data to behavioral events and to conduct chronic electrophysiological recordings has prompted many laboratories to adapt these recording procedures to freely moving animals.

Several different types of electrophysiological signals can be recorded from the brain depending on the type of electrode used to make the recording. Electro-encephalographic (EEG) recordings are made from the outer surface of the skull using large, millimeter-scale electrodes. The large size and low impedance of EEG electrodes, along with the filtering of electrical signals caused by the skull, limits them to recording electrical signals integrated across a large several centimeter sized area of the brain. Electro-corticographic (eCoG) recordings are also made using large, low-impedance electrodes which are place directly on the surface of the brain, i.e. the cerebral cortex. Since the electrodes are placed directly on the surface of the brain they are not hampered by filtering caused by the skull. However, due to their large size and low impedance they still integrate electrical signals over approximately 1-2 centimeters of the brain. Additionally, electrophysiological recordings can be made from within the brain. If within the brain electrophysiological recordings are made using large, low-impedance electrodes, then the electrical signals recorded are similar to eCoG recordings. Alternatively, small micro-scale, high-impedance electrodes can record two electrophysiological signals from within the brain that cannot be recorded using the other techniques. These electrophysiological signals are (1) the action potentials (APs) of individual neurons (sometimes called single-units) and (2) the local field potentials (LFPs), which are currently though to consist of the sub-threshold dentritic currents integrated across approximately several hundred micrometers of brain tissue. It is the APs and LFPs recorded using high impedance (˜0.2-˜2 MΩ) and small conductive surface area (˜10-˜7000 square micrometers) micro-electrodes, or similar technologies, which will be the focus of this patent. In addition to the recording electrodes described above, electrodes from providing electrical stimulation of the brain are commonly implanted in the brain. These Deep Brain Stimulator (DBS) electrodes have been successfully used to treat a variety of neurological motor disorders. However, due to the size and the necessity of being able to pass relatively large electrical currents into the brain, DBS electrode cannot record APs or LFPs.

The key element for making successful AP and LFP recordings from awake, behaving animals is a lightweight and head-mounted microelectrode assembly. Several such devices have been developed over the years. The prior art has also developed devices for use in neuroscience laboratories which perform electrophysiology and also permit simultaneous infusions directly into the recording area. However, previous devices have only been capable of recording data at a single location or only EEG signals. Recently, arrays of micro-electrodes have been developed for recording APs and LFPs from multiple sites in the brain simultaneously. However, the prior art does not describe a device for recording APs and LFPs from multiple sites and at the same time performing local infusion of pharmacological substances. Present designs do not permit direct pharmacological manipulation of the area of the brain from which multi-site AP (action potential) and LFP (local field potential) recordings are being made.

For many decades, animal models have been used for the identification of drugs that ameliorate psychiatric, neuropathological and neuro-degenerative disorders. The principle means of assessing efficacy has been the measurement of behavioral responses. The development of anti-depressant drugs is an excellent example of the successful application of this methodology. Similarly, the development of drugs for the treatment of epilepsy uses behavioral assays of seizure activity. However, behavioral assessment is an indirect measurement of drug effects on neural circuitry. Recent data have shown that electrophysiological signals are modulated by anti-depressant drugs and serve as a predictor of drug efficacy. In addition, the effects of infusing substances into the striatum have been quantified using electrophysiology to understand their relationship to disorders such as Parkinson's disease and schizophrenia. These results suggest that systematic and quantitative electrophysiological screening of pharmaceuticals may prove to be a useful tool in drug development for a variety of neurological and psychological pathologies.

More recently, due to the rapidly developing field of neural prosthetics and brain stimulation a need has arisen to maintain chronic, i.e. several years, electrophysiological contact with neurons in the brain. Currently available, chronically implanted micro-electrode arrays for recording single neural units in neural prosthetic applications lose signals over time. In most cases these micro-electrodes fail completely after being implanted in the brain for several months to a few years. This loss of signal is thought to be primarily due to the inflammatory response engendered by insertion of the microelectrodes into the brain and subsequent relative motion of the microelectrodes and the brain. Even arrays that float with the brain suffer from inflammatory responses that could be ameliorated by a pharmacological intervention.

### BRIEF SUMMARY OF THE INVENTION

In the illustrated embodiment the invention is primary used for screening of novel pharmacological agents for neural effects or efficacy rather than as a direct medical intervention. The invention is also used in basic neuroscientific research. The device is used to test drugs for any neurologically based pathology, e.g. psychosis (schizophrenia), seizure disorders, sleep/arousal disorders. While this device appears to be primarily directed at pathologies of neuro-electrical activity, it may also be useful in testing drugs for diseases such as Parkinson's and Alzheimer's which may also influence AP and LFP activity. Also, in every case of the aforementioned diseases, except for Parkinson's, the etiology is unknown. This device is a valuable scientific tool for understanding the mechanisms of neural pathologies.

An apparatus for simultaneously measuring APs and LFPs in a target tissue and for infusing an agent into the target tissue comprising a body, a cannula mounted on the body, and at least one electrophysiological microelectrode in proximity to the cannula and mounted on the body so that the agent supplied to the cannula is provided to the proximity of the target tissue with which at least one electrophysiological microelectrode is electrically coupled. The cannula and microelectrode are arranged and configured with respect to each in a selected configuration to allow the apparatus to be customized for optimal implantation in specific neurological sites.

The electrophysiological microelectrode is biocompatible and adapted for chronic or acute use. The apparatus further comprises a plurality of such electrophysiological microelectrodes, which are arranged and configured on the body into a predetermined array to record APs and LFPs simultaneously at different sites within the brain so that any changes in APs and LFPs may be quantified in relation to the introduction of a drug through the cannula into the brain. The illustrated embodiment shows a linear array of electrophysiological microelectrodes.

In particular, the illustrated embodiment of the invention is an apparatus for sensing an electrophysiological signal in a target tissue and for infusing an agent into the target tissue. The apparatus comprises a body, a cannula mounted on the body, and a sensing microelectrode, characterized by having an impedance of approximately 0.2-2 MΩ at sensed frequencies when implanted into the target tissue and/or an exposed electrically conductive surface area of approximately ten to several thousand square micrometers, in proximity to the cannula and mounted on the body so that the agent supplied to the cannula is provided to the proximity of the target tissue into which at least one electrophysiological microelectrode is electrically coupled.

The illustrated embodiment of the invention comprises a customized selected arrangement and configuration of the cannula and microelectrode(s) with respect to each other, which allows the apparatus to be customized for a specific neurological site.

The sensing electrophysiological microelectrode is capable of recording electrophysiological action potentials and local field potentials simultaneously in the target tissue.

The sensing electrophysiological microelectrode is biocompatible and adapted for chronic or acute use.

The illustrated embodiment of the invention further comprises a plurality of sensing electrophysiological microelectrodes, each having an impedance of approximately 0.2-2 MΩ at sensed frequencies of interest and/or an exposed electrically conductive surface area of approximately ten to several thousand square micrometers, in proximity to the cannula and mounted on the body so that the agent supplied to the cannula is provided to the proximity of the target tissue with which at least one electrophysiological microelectrode is electrically coupled, the cannula and microelectrode being arranged and configured with respect to each other in a selected configuration to be customized for optimal sensing at multiple specific neurological sites.

The plurality of the sensing electrophysiological microelectrodes are capable of recording electrophysiological action potentials and local field potentials simultaneously on the target tissue.

Each of the sensing electrophysiological microelectrodes of the plurality of sensing electrophysiological microelectrodes is biocompatible and adapted for chronic or acute use.

The plurality of sensing electrophysiological microelectrodes are arranged and configured on the body into a predetermined array.

The predetermined array is a linear, planar, or an arbitrary geometrical array of sensing electrophysiological microelectrodes.

The illustrated embodiment of the invention comprises a microelectrode plate coupled to the body for mounting and positioning the sensing electrophysiological microelectrode.

The illustrated embodiment of the invention comprises a microelectrode plate coupled to the body for mounting and positioning the plurality of sensing electrophysiological microelectrodes into a predetermined array.

The body comprises a manifold for communicating fluid from an external source of the agent to the cannula.

The illustrated embodiment of the invention further comprises a side port defined in the manifold for providing fluidic communication to the external source.

The illustrated embodiment of the invention further comprises an electrical connector coupled to the sensing electrophysiological microelectrode.

The illustrated embodiment of the invention further comprises an electrical connector coupled to the plurality of sensing electrophysiological microelectrodes.

The illustrated embodiment of the invention further comprises an electrical connector mounted on the manifold and coupled to the sensing electrophysiological microelectrode.

The illustrated embodiment of the invention further comprises a plurality of sensing electrophysiological microelectrodes and further comprising an electrical connector mounted on the manifold and coupled to the electrophysiological microelectrode.

The illustrated embodiment comprises an apparatus for sensing an electrophysiological signal in a target tissue and for infusing an agent into the target tissue comprising: a body; a cannula mounted on the body; and a sensing microelectrode characterized by having an exposed, microtip sharpened to approximately 1-2 μm in diameter and 20-50 μm in length, the microtip being positioned in proximity to the cannula and mounted on the body so that the agent supplied to the cannula is provided to the proximity of the target tissue into which at least one electrophysiological microelectrode is electrically coupled.

The illustrated embodiment of the invention also comprises a method comprising the steps of: sensing an electrophysiological signal in tissue with at least one sensing electrophysiological microelectrode characterized by having an impedance of approximately 0.2-2 MΩ at sensed frequencies when implanted into the target tissue and/or an exposed electrically conductive surface area of approximately ten to several thousand square micrometers; and simultaneously infusing an agent into the target tissue though a cannula provided in proximity of the target tissue with which the at least one sensing electrophysiological microelectrode is electrically coupled.

The illustrated embodiment further comprises the step of coupling with a plurality of electrophysiological signals with a corresponding plurality of sensing electrophysiological microelectrodes, each characterized by having an impedance of approximately 0.2-2 MΩ at sensed frequencies when implanted into the target tissue and/or an exposed electrically conductive surface area of approximately ten to several thousand square micrometers.

The step of sensing the electrophysiological signals from the target tissue comprises sensing the electrophysiological signals in a predetermined array in the target tissue.

The step of sensing the electrophysiological signals from the target tissue comprises sensing the electrophysiological signals from the target tissue over a chronic period.

The illustrated embodiment further comprises the step of subcutaneously implanting the apparatus into a subject and telemetering the electrophysiological signal from the target tissue to an external receiver.

The illustrated embodiment further comprises the step of infusing an anti-inflammatory agent in the proximity of the microelectrode to prolong the useful lifespan of the implanted microelectrode to effectively sense the electrophysiological signal.

The step of sensing electrophysiological microelectrode comprises recording electrophysiological action potentials and local field potentials simultaneously in the target tissue.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side elevational view of the implant of the invention.

FIG. 2 is a diagrammatic view of the implant showing the electrical connection of the microelectrodes to the interface.

FIGS. 3a and 3b are micrographs of the immunohistological staining for GFAP showing in FIG. 3a an increased inflammatory response at the site of one of the microelectrodes in comparison with FIG. 3b showing the contralateral hemisphere were no microelectrodes were placed. Animal was sacrificed at 30 days post-apparatus implantation.

FIGS. 4a-4d are graphs of the electrophysiological data collected from the cannula-microelectrode apparatus from two rats (band pass filtered 300-10000 Hz). FIGS. 4a and 4b show multiple APs over the course of one second for rat 2 and 10 s for rat 3. FIGS. 4c and 4d expand the temporal scale to show two single AP discharges. This data was collected at 12 days (rat 3) and 7 months (rat 2) post-array implantation.

FIG. 5 is a graph of the spectral analysis of electrophysiological data collected from the cannula-microelectrode apparatus from one rat (wideband filtered 0.1-10,000 Hz). The LFP exhibits a peak in the power spectrum in the beta and low gamma frequencies (10-50 Hz) typical of recordings from the cerebral cortex. The data was acquired 15 days post-array implantation.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment is a device for assessing the effects of diffusible molecules on electrophysiological recordings from multiple neurons. This device allows for the infusion of reagents through a cannula located among an array of micro-microelectrodes. The device can easily be customized to target specific neural structures. It is designed to be chronically implanted so that isolated neural units and local field potentials are recorded over the course of several weeks or months. Multivariate statistical and spectral analysis of electrophysiological signals acquired using this system could quantitatively identify electrical “signatures” of therapeutically useful drugs.

The invention is a chronically implantable hybrid cannula-microelectrode system 30 for the continuous monitoring of electrophysiological signals during the infusion of chemical and/or pharmacological agents. This system 30 is useful in testing the short-term and long-term effects of drug on electrically active tissues, e.g. the effects of anti-depressant or anti-seizure drugs on neuronal activity in the cerebral cortex.

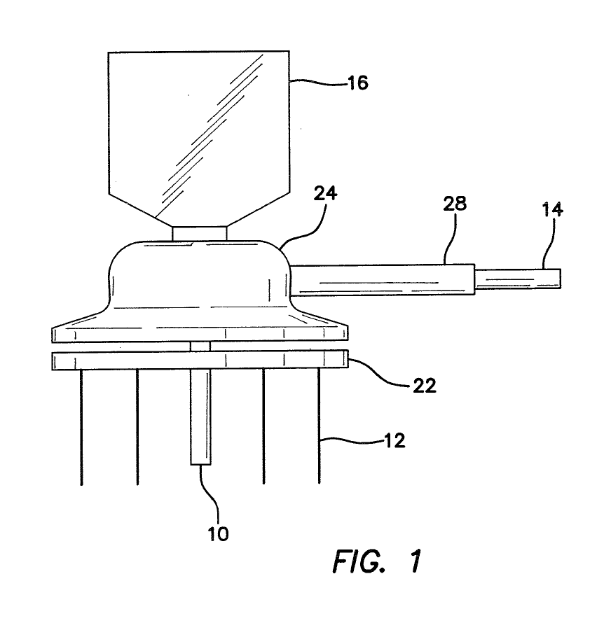

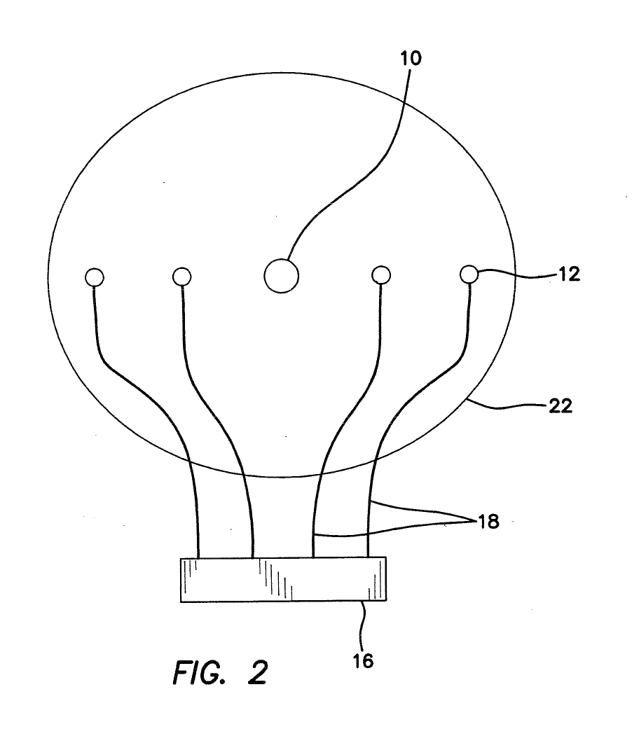

FIG. 1 is a side elevational view of implant system 30 showing a manifold 24 with which a hollow cannula 10 and a side port 28 are communicated. Catheter tubing 14 is coupled to side port 28 so that fluid from an external source can be supplied through side port 28 to manifold 24 and thence to cannula 10. As diagrammatically shown in FIGS. 1 and 2 a cannula 10 is flanked by or associated with a plurality of microelectrodes 12, which are positioned by insulative microelectrode plate 22. The cannula 10 is connected by means of catheter tubing 14 to an infusion device (not shown) such as an osmotic pump, for the delivery of the chemical and/or pharmacological agents.

Microelectrode plate 22 is positioned beneath manifold 24 and provides the mechanical mounting for the array of microelectrodes 12 for recording electrical and local field potentials at several sites at once. The illustrated embodiment depicts four microelectrodes 12, but the number is arbitrary. Further, microelectrodes 12 can be arranged in a plurality of geometric configurations and all of which are within the scope of the invention. FIGS. 1 and 2 illustrate a linear array of microelectrodes 12 by way of example. The microelectrodes 12 are wired through wires 18 to an electrical interface 16 diagrammatically depicted in FIG. 2 and illustrated in side elevational view in FIG. 1 to allow connection to amplifiers, filters, and data acquisition hardware for the recording of electrophysiological signals. Any type of multiple contact electrical connector or telemetry circuit now known or later devised can be provided on interface 16.

In the linear array of FIGS. 1 and 2 cannula 10 is approximately 2.0 mm long and microelectrodes 12 are approximately 2.5 mm long. The diameter of microelectrode plate 22 and manifold 24 is approximately 5.9 mm and the overall height of the device or system 30 from the lower end of microelectrodes 12 to the upper end of interface 16 is approximately 8.8 mm. Clearly other dimensions could have been chosen without departing from the spirit and scope of the invention.

The electrically conductive uninsulated electrode tips are configured specifically for the recording of APs and LFPs. Typically the micro-electrode tips are parabolic in shape with a height of ˜20, a diameter of ˜20 micrometers and an impedance in the range of 0.2-2 MΩ. However, any micro-electrode tip dimensions which enable the recording of APs and LFPs can be chosen with departing from the spirit and scope of the invention.

We have successfully implanted cannula-electrode devices into the frontal and parietal cortexes of rats. Both electrophysiological and histological data was obtained from these animals. The device has proven itself in the acquisition of data in rats. We have both electrophysiological and histological data from several rats used to study the effects of anti-inflammatory drugs on the long-term quality of electrical recordings. The device 30 is surgically implanted through a small opening in the skull, either by making a small burr hole or by craniectomy. The duramater also will be micro-surgically incised prior to implantation or can be pierced by the cannula(i) 10 and microelectrode(s) 12, but is otherwise left intact. The device 30 is anchored to the skull using two titanium screws and small island of surgical acrylic (head-cap). The osmotic pump, which is attached to the device, will also be implanted subcutaneously, while the electrical connector or interface 16 is imbedded in the head-cap. The scalp is sutured closed around the head-cap, leaving the electrical connector 16 exposed. Alternatively, it is possible using wireless telemetry to couple to microelectrodes 12 and to have the entire device 30 installed subcutaneously. A completely subcutaneous installation is advantageous in reducing the risk of infection and discomfort to the animal.

System 30 can also be implanted subcutaneously or surgically implanted into deeper anatomical tissues. System 30 may also be miniaturized and modified using conventional design principles in a manner consistent with the teachings of the invention so that it can be endoscopically implanted into a body. In any case system 30 is usually implanted to allow external access to interface 16 and side port 28.

The free and arbitrary design choices of the cannula, microelectrode number, microelectrode length, and configuration allows the invention to be configured specifically for a biological structure with one or multiple targets. In the illustrated embodiment, the microelectrodes 12 were manufactured from highly biocompatible materials such as platinum, iridium, or Paralene-C. However, microelectrode materials and construction could also be arbitrarily chosen according to the teachings and scope of the invention for different biological structures.

It should be noted that the invention contemplates within its scope the use and implantation of multiple infusion pumps, each with different rates of infusion and/or different agents. In such an embodiment different sets of microelectrodes are associated and operated with operation of the different pumps.

It can now be appreciated that one of the advantages of the invention is the flexibility of its construction. The microelectrode(s) 12 and cannula(i) 10 can be arrange in virtually any configuration, which allows the device 30 to be easily customized for implantation in multiple specific brain areas. Additionally, the design of the invention gives the user the ability to implant the device completely subcutaneously, using telemetry coupled to an external receiver and osmotic pump(s) which are referred to as an external source above. In the case of subcutaneous implantation the source of fluid or agent is external to the device 30, but internal to the animal, i.e. a reservoir (not shown) holding or storing the agent is also implanted. It is also possible the agent or fluid source could also be external to the animal. Finally, the invention allows the user to record both APs and LFPs at the same time within the specified brain regions. Capturing both of these measurements contemporaneously leads to a greater electrophysiological understanding of the brain when a drug is introduced which in turn leads to more effective and efficient drug research. In sum, the device is a highly configurable matrix of microelectrodes 12 and cannuli 10 which is easy to implant both acutely and chronically.

The apparatus of the illustrated embodiment offers a simple and effective way to approach drug development, microelectrode contact longevity issues, and basic neuroscience research. Although several cannula-electrode devices have been designed in the prior art for use in both behaving rats and monkeys, the illustrated embodiment presented here possesses several significant advantages. It its extremely light weight, simple to use, highly configurable, bio-compatible, and can acquire both isolated neural APs and LFPs at multiple sites in the brain, while delivering drugs through a cannula into the area of the brain from which APs and LFPs are being recorded.

The invention having been described in general terms, consider now the details of the assembly of a cannula-multimicroelectrode array. The illustrated embodiment is apparatus 30 for simultaneously measuring electrophysiological signals and for infusing reagents in close proximity to the microelectrodes. As stated above the apparatus as disclosed in FIGS. 1 and 2 is comprised of a body or manifold 24, a cannula 10, and microelectrodes 12 mounted on the manifold 24 so that reagents supplied by the cannula 10 are delivered in proximity of the microelectrodes 12. The cannula 10 and microelectrode 12 can be arbitrarily configured with respect to each other in order to allow the apparatus 30 to be customized for optimal implantation in specific brain regions. The apparatus 30 of FIGS. 1 and 2 is a modification of a commercially available cannula system.

The microelectrodes 12 are made up first, as single long “hat pins”. Holes are drilled at the desired location into one of the microelectrode mounting disks 22 supplied with the Alzet kit. The rigid hat pin microelectrode 12 is placed through the pre-drilled hole with the desired length extending below the microelectrode mounting disk 22 and tacked in place using a small amount of biomedical grade cyanoacrylate glue. The length of microelectrode 12 above the microelectrode mounting disk 22 is trimmed to a shaft of approximately 1 mm and stripped of insulation. A flexible 33 gauge insulated copper wire lead 18 is soldered to the microelectrode shaft 12 so that it is at a right angle to the shaft and parallel to the microelectrode mounting disk 22. The other end of the copper lead 18 can then be attached to any convenient electrical connector 16. The cannula 10 is then slid into the central hole of the microelectrode mounting disk 22, until the desired length of the cannula 10 is protruding below the disk 22, and tacked in place using the cyanoacrylate glue. The gap between the microelectrode mounting disk 22 and the base of the cannula manifold 24 is filled with Loctite M-31CL Medical Apparatus Epoxy to protect wire leads 18 and strengthen the apparatus 30.

The microelectrodes 12 are manufactured from the biocompatible materials, platinum/iridium alloy and provided with a Paralene-C insulation. However, it is to be expressly understood that many other compositions for biocompatible microelectrodes and insulation coatings or films could be substituted. The units tested utilize 75 μm diameter exposed microelectrode tips sharpened to 1-2 μm diameter and 20-50 μm in exposed length after the insulation was removed with impedance of ˜0.3 MΩ. However, microelectrodes of diameters of the order of 10 to 100 μm in diameter with sharpened tips as disclosed above with impedances of the order of 0.2-2 MΩ for frequencies in the range of 0 to 10 kHz are expressly contemplated as within the scope of the invention. The impedance of the microelectrode 12 is primarily dependent on the exposed length and degree or nature of the sharpening of the micro-tip, so that the microelectrode 12 can be equivalently characterized either by its geometric parameters or its impedance at the frequencies of interest. However, specialized electrode surface coatings and treatments can reduce the impedance of a micro-electrode of a given size. The length of microelectrode 12 which is insulated has substantially no effect on its impedance. Only microelectrodes 12 which have been fashioned with an impedances in the range of 0.2-2 MΩ and/or an exposed electrically conductive surface areas of approximately ten to several thousand square micrometers are capable of reliably providing sensed APs and LFPs in neurological tissue. The microelectrodes 12 are used for sensing only, since more than a few tens of microvolts applied to them as a stimulating microelectrode would likely destroy the tip by destroying the insulating layer near the tip or degrading the tip itself and/or destroying the nearby neural tissue, so that microelectrode 12 would then be rendered unable to sense APs or LFPs in neurological tissue thereafter. One aspect of microelectrode 12 prepared as disclosed in the illustrated embodiment is that microelectrode 12 is capable of simultaneously sensing both the action potentials of a single neuron and the local field potential (LFP) of the neurological tissue, which is believed to originate with the nearest neurons, possibly numbering a thousand or more. Action potentials, which have an identifiable profile, are sensed at frequencies in the low kHz ranges whereas LFP's are sensed generally at frequencies of 200 Hz and less. A complex multiple frequency signal is detectable by the modified microelectrode 12 of the illustrated embodiment so that a wide sweep of frequencies are detectable at measureable levels, thereby allowing simultaneous detection of action potentials and local field potentials. The microelectrodes 12 and cannula 10 extended 2.5 mm and 2.0 mm below the microelectrode mounting disk 22 respectively.

Microelectrode materials and construction can also be customized according to the needs for insertion into different brain structures, e.g. longer microelectrodes for recording from deep brain structures. The microelectrode manufacturing and apparatus assembly is carried out by Micro Probe Inc. Using the current version of the apparatus 30, saline is infused using an osmotic mini-pump (not shown). This pump uses the force generated by an osmotic gradient to slowly infuse liquid over the course of several days-to-weeks with no intervention.

Consider now the surgical implantation of apparatus 30. The surgical implantation of the apparatus 30 is performed using a minimally invasive procedure. An extended borehole procedure is performed. The apparatus is then stereotaxically implanted through the craniotomy. The duramater is pierced by the cannula 10 and microelectrodes 12, but is otherwise left intact. The apparatus 30 is anchored to the skull using titanium bone screws and an island of methyl methacrylate forming a small head cap (not shown). A pocket is formed by blunt dissection of a subcutaneous space between the scapulae and an osmotic pump is placed into this pocket and connected to the cannula-microelectrode apparatus 30 with plastic tubing. The scalp is sutured around the headcap, leaving the electrical connector 16 exposed. A skilled operator can implant the apparatus in approximately 20 min from the onset of anesthesia.

It has been reported that cyanoacrylate gel (loctite 454) is a more effective and easier means of cannula-microelectrode fixation since it does not require the use of skull screws for anchoring. This would greatly reduce the time required for implantation.

Consider the data acquisition and analysis. Electrophysiological data can be acquired using standard amplification, filtering, and analog to digital converting systems. We recorded isolated APs and LFP using two signal paths and with different filters applied to each path. We used a Dam-80 isolation amplifier and filter and a National Instruments DAQ card. Electrical signals are amplified with a gain of 10 k and filtered at either 100-10,000 Hz for recording APs, or 0.1-10,000 Hz to acquire LFPs. Alternatively, a single broadband neural signal could be recorded and differentially digitally filtered offline.

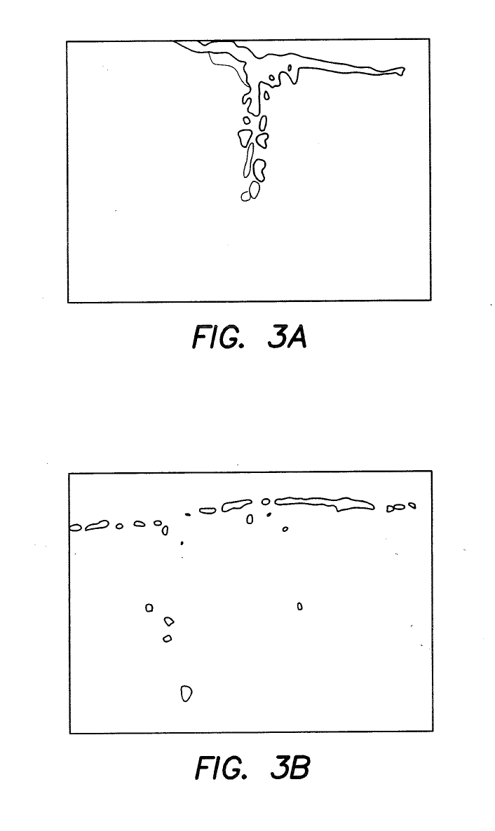

We successfully implanted this apparatus into the frontal or parietal cortices of five rats, and obtained both electrophysiological and histological data. Activated astrocytes are a key part of the inflammatory response to neural injury, and increased GFAP staining is a reliable maker of this response. Several weeks post-implantation, we sacrificed the rats and performed GFAP immunohistochemistry. As expected, compared to the non-implanted hemisphere, the tissue around the microelectrode 12 exhibits increased GFAP immunostaining as shown in FIG. 3a as compared to FIG. 3b.

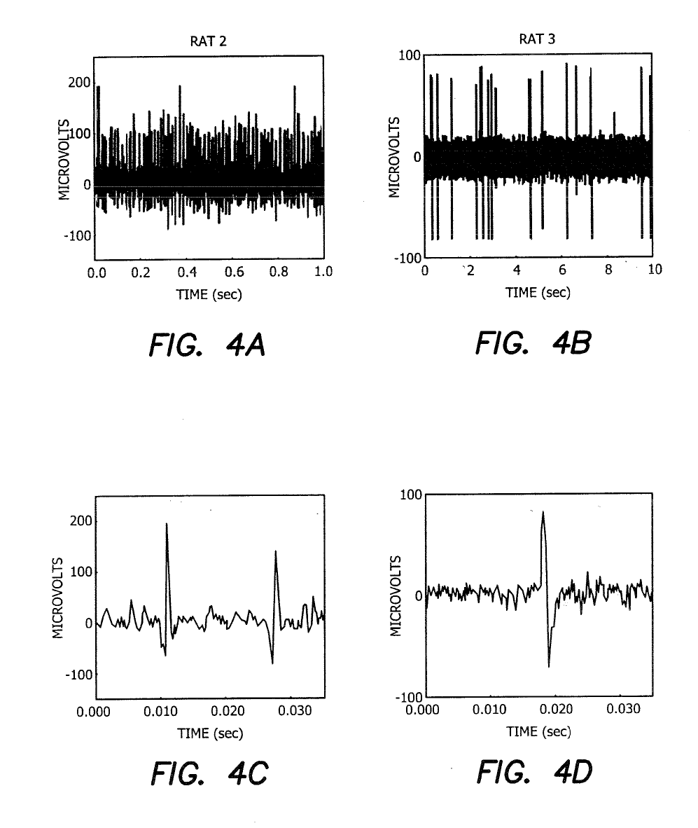

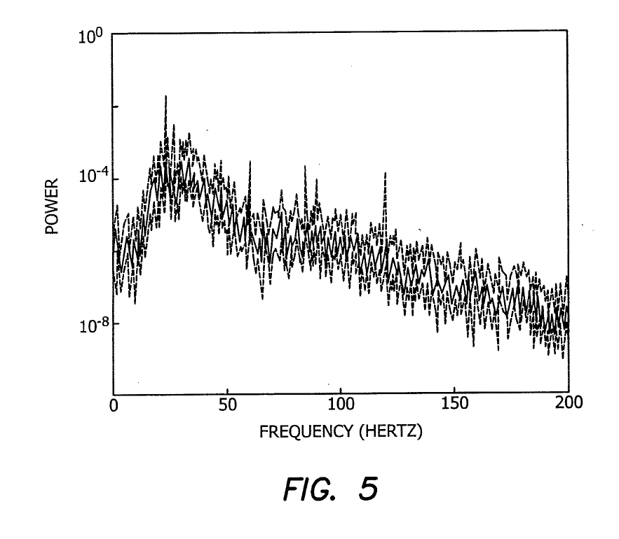

We also collected electrophysiological data at two to five time points over many weeks post-implantation as illustrated in the graphs of FIGS. 4a-4d and 5. Even though an increase in the inflammatory response was detected by imunohistochemistry, we are able to collect high quality electrophysiological data. As calculated by spike peak-to-peak divided by the RMS of the whole recording, the signal to noise ratio of the recordings displayed in FIGS. 4a-4d is 19:1 for rat 2 and 25:1 for rat 3. Both the high frequency spike data and the spectral analysis of the LFP demonstrate electro-physiological activity 2 weeks post-implantation is shown.

The cannula-microelectrode apparatus 30 described here allows recording of the electrical signal from single neural units, and the more global LFP signal, at multiple sites. The recordings of electrical activity are made while a reagent is infused in close proximity to the recording microelectrodes. Similar apparatus used by others are capable of recording at only a single location, or only EEG signals. The present apparatus is highly configurable so that electrical recordings and reagent infusion can be targeted to specific neural structures.

We recorded electrical activity from, and infused saline into, the cerebral cortex, which served as a proof of concept for the functionality of the apparatus. In addition, since cytokines such as interleukin (IL)-1, -4, -8, -10 and tumor necrosis factor-α (TNF-α) can enhance repair of injured tissue, it is contemplated that use of the described cannula-microelectrode apparatus 30 in testing such anti-inflammatory agents will determine which particular anti-inflammatory agent will prolong the useful lifespan of the microelectrode arrays to the greatest extent. Thus, apparatus 30 could serve as a tool for determining pharmaceutical methods of improving the longevity of chronically implanted microelectrodes used in neural prosthetic applications.

Recent studies have shown that electrophysiological signals from isolated neurons are affected by neuroactive drugs or anti-depressants and that evoked potential responses can serve as a marker of anti-depressant efficacy. Such results suggest that there are likely to be electrophysiological signatures for neuro-active drugs effective against a variety of neuro-pathologies. Recordings of APs and LFPs may allow for the detection of such signatures in localized neural structures. The effects of intra-cerebral infusion of pharmaceutical agents could then be examined for their effects upon electrophysiological signatures.

When coupled with telemetry for wireless transmission of the neural signals, there is no need for a transcutaneous electrical connector, so the skin can be sutured completely closed over the acrylic head-cap. In such a configuration the apparatus could provide continuous infusion of reagents and monitoring of signals in the freely behaving animal without requiring a wired connection and a commutator.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for sensing an electrophysiological signal in a target tissue and for infusing an agent into the target tissue comprising:
a body;
a cannula mounted on the body; and
a sensing microelectrode, characterized by having an impedance of approximately 0.2-2 MΩ at sensed frequencies when implanted into the target tissue and/or an exposed electrically conductive surface area of approximately ten to several thousand square micrometers, in proximity to the cannula and mounted on the body so that the agent supplied to the cannula is provided to the proximity of the target tissue into which at least one electrophysiological microelectrode is electrically coupled.

2. The apparatus of claim 1 further comprising a customized selected arrangement and configuration of the cannula and microelectrode(s) with respect to each other, which allows the apparatus to be customized for a specific neurological site.

3. The apparatus of claim 2 where the sensing electrophysiological microelectrode is capable of recording electrophysiological action potentials and local field potentials simultaneously in the target tissue.

4. The apparatus of claim 2 where the sensing electrophysiological microelectrode is biocompatible and adapted for chronic or acute use.

5. The apparatus of claim 1 further comprising a plurality of sensing electrophysiological microelectrodes, each having an impedance of approximately 0.2-2 MΩ at sensed frequencies of interest and/or an exposed electrically conductive surface area of approximately ten to several thousand square micrometers, in proximity to the cannula and mounted on the body so that the agent supplied to the cannula is provided to the proximity of the target tissue with which at least one electrophysiological microelectrode is electrically coupled, the cannula and microelectrode being arranged and configured with respect to each other in a selected configuration to be customized for optimal sensing at multiple specific neurological sites.

6. The apparatus of claim 5 where the plurality of the sensing electrophysiological microelectrodes are capable of recording electrophysiological action potentials and local field potentials simultaneously on the target tissue.

7. The apparatus of claim 5 where each of the sensing electrophysiological microelectrodes of the plurality of sensing electrophysiological microelectrodes is biocompatible and adapted for chronic or acute use.

8. The apparatus of claim 5 where the plurality of sensing electrophysiological microelectrodes are arranged and configured on the body into a predetermined array.

9. The apparatus of claim 8 where the predetermined array is a linear, planar, or an arbitrary geometrical array of sensing electrophysiological microelectrodes.

10. The apparatus of claim 1 further comprising a microelectrode plate coupled to the body for mounting and positioning the sensing electrophysiological microelectrode.

11. The apparatus of claim 5 further comprising a microelectrode plate coupled to the body for mounting and positioning the plurality of sensing electrophysiological microelectrodes into a predetermined array.

12. The apparatus of claim 1 where the body comprises a manifold for communicating fluid from an external source of the agent to the cannula.

13. The apparatus of claim 12 further comprising a side port defined in the manifold for providing fluidic communication to the external source.

14. The apparatus of claim 2 further comprising an electrical connector coupled to the sensing electrophysiological microelectrode.

15. The apparatus of claim 5 further comprising an electrical connector coupled to the plurality of sensing electrophysiological microelectrodes.

16. The apparatus of claim 12 further comprising an electrical connector mounted on the manifold and coupled to the sensing electrophysiological microelectrode.

17. The apparatus of claim 12 further comprising a plurality of sensing electrophysiological microelectrodes and further comprising an electrical connector mounted on the manifold and coupled to the electrophysiological microelectrode.

18. A method comprising:
sensing an electrophysiological signal in tissue with at least one sensing electrophysiological microelectrode characterized by having an impedance of approximately 0.2-2 MΩ at sensed frequencies when implanted into the target tissue and/or an exposed electrically conductive surface area of approximately ten to several thousand square micrometers; and
simultaneously infusing an agent into the target tissue though a cannula provided in proximity of the target tissue with which the at least one sensing electrophysiological microelectrode is electrically coupled.

19. The method of claim 18 further comprising coupling with a plurality of electrophysiological signals with a corresponding plurality of sensing electrophysiological microelectrodes, each characterized by having an impedance of approximately 0.2-2 MΩ at sensed frequencies when implanted into the target tissue and/or an exposed electrically conductive surface area of approximately ten to several thousand square micrometers.

20. The method of claim 19 where sensing the electrophysiological signals from the target tissue comprises sensing the electrophysiological signals in a predetermined array in the target tissue.

21. The method of claim 19 where sensing the electrophysiological signals from the target tissue comprises sensing the electrophysiological signals from the target tissue over a chronic period.

22. The method of claim 18 further comprising subcutaneously implanting the apparatus into a subject and telemetering the electrophysiological signal from the target tissue to an external receiver.

23. The method of claim 18 further comprising infusing an anti-inflammatory agent in the proximity of the microelectrode to prolong the useful lifespan of the implanted microelectrode to effectively sense the electrophysiological signal.

24. The method of claim 18 where the sensing electrophysiological microelectrode comprises recording electrophysiological action potentials and local field potentials simultaneously in the target tissue.

25. An apparatus for sensing an electrophysiological signal in a target tissue and for infusing an agent into the target tissue comprising:
a body;
a cannula mounted on the body; and
a sensing microelectrode characterized by having an exposed, microtip sharpened to approximately 1-2 μm in diameter and 20-50 μm in length, the microtip being positioned in proximity to the cannula and mounted on the body so that the agent supplied to the cannula is provided to the proximity of the target tissue into which at least one electrophysiological microelectrode is electrically coupled.
